(19) United States
(12) Patent Application Publication
(10) Pub. No.: US 2004/0086888 A1
(43) Pub. Date: May 6, 2004

(54) Method for tandem genomic/proteomic analysis of proliferating cells

(76) Inventors: Paul L Kornblith, Pittsburgh, PA (US); Maryann Donovan-Peluso, Pittsburg, PA (US)

Correspondence Address: Patent Administrator, Testa Hurwitz & Thibeault, High Street Tower, 125 High Street, Boston, MA, 02110, US

(21) Appl. No.: 10/399,563

(22) PCT Filed: Oct. 18, 2001
(86) PCT No.: PCT/US01/32540

(51) Int. Cl. C12Q 1/68; C12Q 1/66; G06F 19/00; G01N 33/48; G01N 33/50

(52) U.S. Cl. 435/6; 702/20; 435/8

(57) ABSTRACT

A novel process for producing data characterizing nucleic acids in proliferating cells using a tandem protocol for simultaneously building and expanding genomics/proteomics cancer databases while coordinating present and future genomic/proteomic individual patient assays by collecting a tissue sample including the proliferating cells; mechanically dividing the sample into cohesive multicellular particulates, and growing and analyzing the resulting cells. Sets of genetic data can be analyzed along with sets of corresponding clinical data, including phenotypic data, to form profiles that can aid in identifying proliferative diseases and in prognoses. A method for diagnosing proliferative diseases using the described assay methods is also provided. Lastly, a computing device is provided which permits searching analysis and entry of the genetic data, the corresponding clinical data and/or profiles.

### BACKGROUND OF THE INVENTION

Provided is a novel process for producing data characterizing the nucleic acids in proliferating cells using a tandem protocol for simultaneously building and expanding genomics/proteomics cancer databases while coordinating present and future genomic/proteomic individual patient assays that allows for diagnostic and prognostic evaluation of proliferative diseases.

Understanding the relationship between genetic markers and/or events and the progression of a proliferative disease (i.e., a disease or condition, such as cancer or psoriasis, in which cells proliferate in an abnormal fashion) traditionally has proven to be a very slow process. Typically, an aberrant or aberrantly expressed gene or gene product is identified and, through a variety of well-known methodologies, the sequence of the gene may be determined and the expression of the gene may be evaluated. Through laborious experimentation and exploration, the relationship between expression of a gene and treatments/outcomes may be determined.

Many technologies have been developed which take advantage of such relationships to diagnose certain proliferative diseases. These technologies often involve the evaluation of nucleic acids in a biopsy of a proliferating tissue. The expression levels of a particular gene and/or the presence of polymorphisms may be determined. The methods for evaluating the nucleic acids of the proliferating tissue are many and include, without limitation, hybridization studies (i.e., Southern and Northern blots); nucleic acid sequencing; fingerprinting (i.e., the analysis of restriction fragment length polymorphisms); and PCR-based protocols, which may be quantitative and/or qualitative in nature. These protocols may be performed in a traditional manner, i.e., by running the results on a gel, or by newer methods. These newer methods involve miniaturization and multiplication of the traditional protocols and include nucleic acid microarrays and a variety of applications that may be performed in connection with such microarrays (i.e., without limitation, DNA:DNA or DNA:RNA hybridizations and/or competitive hybridizations). The arrays permit the rapid analysis of a nucleic acid sample for the presence or absence of 12,000 or more independent nucleic acids. These independent nucleic acids may be of known genes or of unknown genes [i.e., expressed sequence tags (ESTs)].

One particular difficulty in using the above-described assays to identify the altered expression of or sequence of a given gene or other target DNA in proliferating cells in a tumor is that in most cases the proliferating cell is mixed in with other normal cells, such as fibroblasts. Typically, the actual proliferating cell comprises less than 5% of the cell population in a given biopsy. In assays that analyze sequence differences between normal and proliferating cells, if the differences between the two DNAs are large enough, the normal and proliferative nucleic acids may be distinguished. However, more subtle differences may not be distinguishable from the background of normal nucleic acids.

This problem is compounded in assays that quantify RNA (expression) levels. Only the most macroscopic of deviations in quantities of RNA ate detectable when the deviant RNAs form only a small percentage (i.e., 0.1-5%) of a mixed pool (population) of RNAs. “Subtler” deviations in RNA levels will remain undetectable. In this case, a “subtler” deviation may actually be a gross deviation, such as a 1000% increase in the expression of a particular gene. For example, a mixed pool of RNA, a 1000% increase in the quantity of a given RNA species 2% of the cells in a biopsy will appear as a 20% increase in the presence of a given RNA. Similarly, a 100% increase in transcription of a given gene would result in only a 4% increase in overall RNA levels in the mixed population. These deviations (4-20%) may be undetectable, or at least statistically insignificant, in many assay systems. This difficulty prevents the realization of the power of certain combinatorial and/or miniaturized assays, such as microarray technologies.

Thus, there is a substantial need to isolate substantially pure populations of proliferating cells from “background,” i.e., normal, cells in order to provide nucleic acid populations that are suitable for conducting statistically significant genetic analysis of the cells. The isolation of substantially pure populations of such cells would not only provide a powerful means for identifying genetic markers specific to any given proliferative disease, but when applied to individual patients would be a powerful diagnostic and outcome-predicting (prognostic) tool. This would permit the realization of the power of combinatorial and/or miniaturized genetic assays, such as microarray technologies, and the computerized analysis thereof.

Non-genetic analysis, i.e. phenotypic analysis, of the substantially pure population of cells derived from a tissue sample also is of value in the diagnosis of proliferative diseases, the resultant non-genetic data can be combined with the above-described genetic data to provide a complete and accurate profile of the proliferative cells of the tissue sample.

### SUMMARY OF THE INVENTION

The present invention is directed to a novel process for producing data characterizing the nucleic acids in proliferating cells using a tandem protocol for simultaneously building and expanding genomics/proteomics cancer databases while coordinating present and future genomic/proteomic individual patient assays. The protocol is comprised of culturing proliferating cells by collecting a tissue sample including the proliferating cells; mechanically dividing the sample into cohesive multicellular particulates; and growing a tissue culture monolayer from the multicellular particulates. Such monolayers can be frozen for future analytic and/or prognostic and diagnostic investigations. The genetic data thus generated is then gathered to form a data set in a data structure that also includes data identifying the source of the tissue sample. Clinical data specific to the patient from whom the tissue sample was taken may also be included in the data set.

Purification and classification of nucleic acids can be performed on any nucleic acid that is present in the proliferating cells, i.e., RNA or DNA. Analyses typically involve use of one or more analytical methods known to molecular biologists to characterize the nucleic acids, including, without limitation, quantitative methods that identify the amount of specific RNAs in a cell, and qualitative methods that determine the presence or absence of specific genetic markers, such as DNA or RNA sequence insertions, deletions or substitutions.

One important embodiment of the present invention involves the use of genomic and proteomic dynamic assays to analyze tumor cells grown in culture.

The method may further include the step of characterizing the proliferating cells by analyzing the genetic data in connection with a set of corresponding clinical data for statistically significant commonalities and/or trends. The genetic/clinical correlative data can be used to generate one or more profiles which link one or more proliferative cell disease states with phenotypic and/or genotypic characterizations, diagnoses and/or prognoses. The data and/or profiles may be encoded in a computer storage medium. A method for diagnosing proliferative diseases is also provided that compares either 1) the genetic and corresponding clinical data and/or 2) the profiles generated therefrom, to data generated in connection with a new tissue sample. A computer system containing the data and/or profiles is also provided that, optionally, allows dissemination and/or analysis of the data over a computer network.

### BRIEF DESCRIPTION OF THE DRAWINGS

FIGS. 1A-1C are graphs of the growth rates of three independent cell cultures;

FIGS. 2A-2F through FIGS. 5A-5F are graphs depicting the results of short-term and long-term chemotherapy assays as follows:

FIGS. 2A-2F and FIGS. 3A-3F show short-term and long-term assays for a first patient;

FIGS. 4A-4F and FIGS. 5A-5F show short-term and long-term assays for a second patient;

FIGS. 6 and 7 show two radiation dose versus surviving fraction curves for two glioblastoma cell lines. Cells were irradiated in microtiter plates and assayed four days post-irradiation;

FIGS. 8A-8C are graphs of survival rates of cell cultures treated with radiation (FIG. 8A) or with radiation and Taxol (FIGS. 8B and 8C);

FIGS. 9A and 9B are graphs showing data from a series of experiments where target cells from two tumor types were exposed to Activated Natural Killer (ANK) cells; and

FIG. 10 is a schematic diagram of a computer network for sharing the data generated by the methods of the present invention.

### DETAILED DESCRIPTION OF THE PREFERRED EMBODIMENTS

Other than in the operating examples, or where otherwise indicated, all numbers or expressions referring to quantities of ingredients, reaction conditions, etc. used in the specification and claims are to be understood as modified in all instances by the term “about.”

The present invention is a novel process for producing data characterizing the nucleic acids in proliferating cells using a tandem protocol for simultaneously building and expanding genomics/proteomics cancer databases while coordinating present and future genomic/proteomic individual patient assays, with a two-way feedback system that enhances each. This tandem genomic/proteomic concept consists of two initiatives that proceed side-by-side both at a first point in time and again at a later point in time with respect to samples of malignant tissue from a specific patient or patients.

The process includes the steps of selectively expanding a population of proliferating cells from a tissue sample, typically from a solid tumor, and subsequently extracting nucleic acid from cells of the expanded population. Characterization of the nucleic acid is performed according to the plethora of analytical techniques available to molecular biologists for quantitatively and/or qualitatively analyzing the nucleic acids.

The tandem genomic/proteomic protocol consists of taking the malignant cells of interest at point “A” in time to create a gene relationship database in which the generation of genetic data can be used to determine which gene or gene product relates to the response of a given agent. Microarray assays are then performed to generate genetic data that is correlated to clinical cell lines in order to develop the database. Gene assays are then performed in which antisense is produced for each gene or gene product associated with a given response. Production of antisense is then used as a reagent in a micro version of the cell-based assay system on cells that have been preselected as being representative of the original tumor, based on the fluorescent staining for the cell types and subtypes. Such micro version assays can be used for patient diagnostic and prognostic purposes. Because the cells grown in the monolayer can be frozen for later use, the same genomic/proteomic database creation may be expanded at a later point “B” in time. Also, at point “B”, a micro version genomic/proteomic assay of the patient cells for patient diagnostic and prognostic purposes may be conducted again. The comparison of the database and the patient assay may be conducted at point “A” in time, may be conducted at point “B” in time, and a further timeline comparison may be completed when all the data for points “A” and “B” are available. The present invention also allows for a cellular-based live cell dead cell real time assay system using an ATP bioluminescence assay to determine tumor cell apoptosis levels based on ATP-ADP ratios.

The genomic/proteomic tandem protocol generates data by the analytical process of the present invention (hereinafter the “genetic data”) that is then gathered to form a data set in a data structure that also includes data identifying the source of the tissue sample (the “sample source identifier,” which is herein included in the meaning of the phrase “genetic data”). Clinical data specific to the patient from whom the tissue sample was taken also may be included in the data set. The nature of the clinical data may vary, but it is preferred that the clinical data is more, rather than less, complete. Clinical data may include, without limitation, general patient information, such as age, sex, height, weight, medical history, geographic area(s) of residence, travel habits, sexual activity and ethnicity. Clinical data may also include, without limitation, data specific to the current medical state of the patient, such as symptoms; current and past medications; the quantity administered to the patient and the duration of treatment; other non-pharmaceutical treatments, such as surgery or radiation therapy; data indicating the size and number of tumors and their location in the patient; and other clinical data that indicate the general state of health and/or progression of the proliferative disease state in that patient, such as the outcome of treatments.

The clinical data also can include data generated in the analysis of cells expanded according to the methods of the present invention for sensitivity to drugs. Data indicating the phenotype of the proliferating cells also may be included. This phenotypic data includes, without limitation, histochemical, immunohistochemical, biochemical and growth characteristics of the cells and/or tumor, including the production of secreted compounds, whether or not the proliferative cells were cultured according to the methods of the present invention. Phenotypic data that can be used to identify a particular proliferative disease state is referred to herein and in the appended claims as “phenotypic polymorphisms characteristic of a proliferative disease state.” The growth and analysis of a substantially pure population of proliferating cells of the present invention for drug or radiotherapy sensitivity and for both cellular and secreted phenotypic markers is described herein and in co-pending U.S. patent application Ser. No. 09/189,310, filed Nov. 10, 1998, which is incorporated by reference, providing a comprehensive and unified system for monitoring a malignancy in a patient through the duration of the malignancy.

The cell culture method of the present invention permits the expansion of a population of proliferating cells in a mixed population of abnormal proliferating, cells and other (normal) cells. The mixed population of cells typically is a biopsy or sample from a solid tumor. In the culture method of the present invention, a tissue sample from the patient is harvested, cultured and analyzed for genetic markers. Subcultures of the cells produced by the culture methods of the present invention may be separately exposed to a plurality of treatments and/or therapeutic agents for the purpose of objectively identifying the best treatment for the cultured cells obtained from the patient. The culture techniques of the present invention also result in a monolayer of cells that express cellular markers, secreted factors and tumor antigens in a manner representative of their expression in vivo. Specific method innovations, such as tissue sample preparation techniques, render this method practically, as well as theoretically, useful. One particularly important tissue sample preparation technique is the initial preparation of cohesive multicellular particulates of the tissue sample, rather than enzymatically dissociated cell suspensions or preparations, for initial tissue culture monolayer preparation. With respect to the culturing of abnormal proliferating cells, for example, malignant cells, it is believed (without any intention of being bound by the theory) that by maintaining the abnormal proliferating cells within a multicellular particulate of the originating tissue, growth of the abnormal proliferating cells is facilitated versus the overgrowth of fibroblasts or other cells which tends to occur when suspended tumor cells are grown in culture. Practical monolayers of cells may thus be formed to enable meaningful analysis of the nucleic acids of the cells, screening of a plurality of treatments and/or agents, the identification of cellular markers, and for use in a wide variety of applications, including pathology and vaccine preparation.

One important embodiment of the present invention involves the use of dynamic assays to analyze the tumor cells grown in culture. Proteomic dynamic assays may include, without limitation, functional assays to quantitate protein activity (e.g., presence or absence of urokinase or MMP2); and tumor cell protein expression using medium-based assays for angiogenesis factors, tumor markers, and growth factors or soluble growth factor receptors. Genomic dynamic assays may include, without limitation, gene expression profiling and pharmacogenomic analysis of individual tumor expression profiles using microarray analysis or expression PCR; gene mutation analysis using PCR-sequence analysis; gene therapy that includes herpes-TK ganciclovir suicide gene, p53 transduction effect on chemoresponse and antisense therapy; and gene expression profiling in pre- and post-treatment cells so as to correlate tumor chemoresponse with genotype. Cell phenotype functional dynamic assays may include, without limitation, tumor cell phenotype assays, such as adhesion, migration, chemotaxis and invasion analysis and functional assays to evaluate new cytostatic therapeutics for chronic disease management, such as herceptin effects on HER2/neu protein levels in breast cancer, anti-adhesion peptides, anti-angiogenesis peptides and antibodies, and anti-protease anti-invasion peptides and antibodies. Finally, combination proteomic/genomic dynamic assays may include, without limitation, single cell analysis, such as characterization of individual cells, as well as tumor populations and high-throughput multiparameter screens.

Drug assays can be used to monitor the growth of cells in order to ascertain the time to initiate the assay and to determine the growth rate of the cultured cells; sequence and timing of drug addition is also monitored and optimized. By subjecting uniform samples of cells to a wide variety of active agents (and concentrations thereof), the most efficacious agent can be determined. For assays concerning cancer treatment, a two-stage evaluation is contemplated in which both acute cytotoxic and longer-term inhibitory effects of a given anti-cancer agent are investigated.

With regard to the initial culturing of multicellular particulates, it is believed (without any intention of being bound by the theory) that because the abnormal proliferating cells are grown, under such conditions, closer to that which is found in vivo, the cells express cellular markers, secreted factors and tumor antigens in a manner more closely resembling their expression in vivo. By assaying the culture media obtained from growing a monolayer according to the inventive method or by histochemically and/or immunohistochemically assaying the cells grown, under such conditions, a more accurate profile of the cellular markers or factors is obtained.

The data may be analyzed without the aid of a computer. In certain instances, it will be clear from the data that a certain genetic defect is present in the diseased proliferating cells. For instance, the over-expression of one or more genes that is characteristic of a certain disease state may be readily visualized. However, the strength of the process of the present invention lies in the use of computers to analyze the genetic data in connection with the clinical data and as an aid in diagnosis or prognosis. Therefore, the genetic and clinical data typically are stored in a computer data structure (database) for analysis and/or extraction at a later date.

It is preferred that the above-described data structure is encoded in a computer storage medium in a form that allows searching of the data, input of additional data and/or data mining. Typically, the data structure forms a part of a database that is searchable by use of one or more of the large number of database search engines that are broadly available. The searches may be structured to limit the access of users of the database to specific types of searches, or unstructured, so that end users may define searches that are limited only by the types of searches available in the search engine being used.

The data also may be made available in a form that permits data mining. “Data mining” (Knowledge Discovery in Databases or KDD) is typically defined as the nontrivial extraction of implicit, previously unknown, and potentially useful information from data. The above-described process for establishing relations between the genetic data and the clinical data is a form of data mining since new relationships are discovered by this process. It uses machine learning, statistical and visualization techniques to discover and present knowledge in a form that is easily comprehensible to humans. Data mining typically is expedited by the use of computers and may be performed by the use of one or more of the large variety of commercially available software programs that enable computerized data mining. Standard database search engines may be used for data mining, but programs are available that are much more efficient and capable.

Access to the data and/or database encoded on the computer storage medium may be on a single-user personal computer (PC) or workstation. The data and/or database also may be accessed over a computer network of two or more PCs or workstations. By “computer network” it is meant any connection between two computing devices by which data can be shared or transferred. Computer networks include, without limitation, remote direct connections, by modem or otherwise; local area networks (LANs); intranets; extranets (wide area networks, or WANs); the Internet (including the World Wide Web) or other worldwide computer networks. Also included in the definition of“computer network” are computer file sharing communities that allow location of and sharing of data files over large computer networks, such as the Internet. Examples of such communities are, without limitation, the Napster (client/server), Gnutella and FreeNet (peer-to-peer) paradigms. The term “computer” is to be given its broadest interpretation, including without limitation, PCs, workstations, mainframes, servers and computing appliances.

As used herein, the phrase “computer storage medium” and the like includes any device, internal or external, permanent or removable, in which data can be stored, either temporarily or permanently, for use in connection with a computer. Permanent storage media includes magnetic-, optical- or hardware-based media. Examples of magnetic media include, without limitation, hard drives, floppy drives, magnetic tapes and other removable magnetic media, i.e., zip drives. Optical media include, without limitation, CD-ROM, CD-R, CD-RW, DVD-ROM and DVD-RAM media. Hardware-based media include conventional RAM (i.e., temporary storage of the data in a computer's volatile RAM), ROM and other firmware that can store the data, either temporarily or permanently, and is permanently or removably stored in connection with a computing device.

In one embodiment of the present invention, raw genetic and clinical data is encoded in a computer storage medium in a form that permits mining of the data or otherwise permits interpretation of the data. Once a statistically significant number of records (sets of data corresponding the genetic and clinical data for a single tissue sample) are entered into the computer, generalities may be made about the data and profile(s) may be generated and stored in searchable form. For instance, there may be enhanced expression of a particular gene (i.e., EGFR) in tumors of a certain origin and having certain phenotypic characteristics (i.e., the presence of thrombogenic factors). From the data, and as a non-limiting example, it may be determined that persons from whom a particular cell type is taken have a 50% chance of survival after 6 months with treatment with a chemotherapeutic agent A and a 100% survival rate after 2 years with agent B. The creation of statistically significant profiles enables production of a database which can be searched by physicians and clinical laboratory personnel to aid in diagnoses and prognoses.

The data may be updated anytime new specimens are analyzed. The data may be entered remotely by end users or at a central location by a person or organization that maintains the database. Entry of the new data can be accomplished by a variety of software interfaces, typically customized database software, that permits entry of new records into a database. These interfaces are so common and variable in structure that they are not described herein in further detail. For instance, an end user may manually enter clinical data into the database, while the raw data from a microarray reader is entered automatically into the database to lessen the likelihood of data manipulation or errors in the communication of the data to the database. The broad availability of the database including microarray data derived from cells cultured according to the methods of the present invention will permit rapid compilation of statistically significant and meaningful data for a large number of proliferative disease states. The analyzing step may be either partially or fully automated by robotic devices.

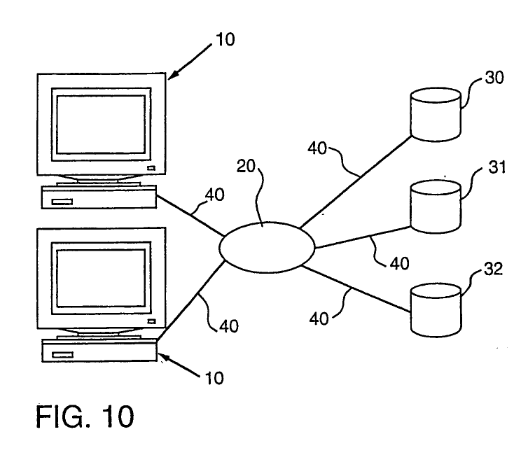

One embodiment of the computer network is shown schematically in FIG. 10. The network includes a plurality of workstations 10 in data communication with a process 20 which is in data communication with data sets 30, 31 and 32. The data sets 30, 31 and 32 include a data set 30 containing genetic data; a data set 31 containing clinical data corresponding to the genetic data; and a data set 32 containing profiles derived from the data mining of data sets 30 and 31. Data communication pathways 40 connect the data sets 30, 31 and 32 with the process 20 and the workstations 10. Process 20 and/or data sets 30, 31 and 32 are optionally contained within any one or distributed between all of workstations 10, and/or in a separate device, such as a server and/or in data storage devices attached to or housed within the workstations 10 and/or server. Workstations 10 can be any computing device having a display and a data input means, such as a keyboard and mouse. Process 20 is performed by a computer and includes data search, data input and analysis functions. The functions are performed by software and/or hardware structures in the computer. Data in data sets 30 and 31 are analyzed by a data mining function of process 20 to generate profiles that link genetic and clinical data which are stored in data set 32. Data communication pathways 40 are any pathway by which data can be transferred, including electrical, electromagnetic and optical pathways.

Central to the present invention is the method for selecting a population of proliferating cells from a tissue sample or biopsy. As described above, prior art methods of analyzing the nucleic acids from a tissue sample are insensitive because the abnormal proliferating cells comprise such a small percentage of the total number of cells in a typical solid tumor. By selectively expanding the abnormal proliferating cells to the exclusion of other cell types, the sensitivity of genetic tests, especially those that quantify expressed sequences (specific RNAs), is increased to the point that the data generated by such methodologies is meaningful.

Thus, an integrated, comprehensive, cellular, molecular and genetic guide to individualized therapy through the duration of the malignancy and thereafter is provided. The method of the present invention also allows for initial identification, tracking, and analyzing of an individual patient's malignancy; identification of malignancy-specific cellular or secreted markers and of cellular or secreted markers indicative of complications; study of the invasiveness and aggressiveness of the malignancy; study of the growth rate of the malignancy; study of the effect of therapies on the malignancy as compared to control cells of the same patient (chemosensitivity and/or radiosensitivity versus theratoxicity) and the identification of a therapeutic index (i.e., the ratio of chemosensitivity:theratoxicity); study of tumor morphology; and study of histological, cytochemical and immunocytochemical markers. The method of the present invention also permits accurate genetic analysis of malignant cells as well as the gathering, analysis, and dissemination of data specific to a given patient. Finally, an integrated system is presented for deciding which drugs to study in specific patients for use in clinical trials for pharmaceutical companies.

The method of the present invention includes the steps of collecting a tissue sample or specimen of a patient's cells and separating the specimen into cohesive multicellular particulates (explants) of the tissue sample, rather than enzymatically digested cell suspensions or preparations. The cells are then grown as a tissue culture monolayer from the multicellular particulates to form a prime culture. A specimen can be taken from a patient at any relevant site including, but not limited to, tissue, ascites or effusion fluid. Samples may also be taken from body fluid or exudates as is appropriate. A tissue culture monolayer can be grown in any method known in the art for growing such a monolayer, for instance, in tissue culture plates or flasks.

Once a prime culture is established from a patient's abnormal proliferating cells, the prime culture can be maintained without any treatment besides normal feedings and passage techniques as indicative of the growth of the cells absent treatment with a therapeutic regimen. Subcultures of the prime culture are prepared so that the cells of the prime culture are not affected by any subsequent testing or treatments. Although prime culture is preferably left untreated, either the prime culture or a subculture thereof can be propagated as a reference culture. The reference culture is a culture which is treated with therapies reflective of a patient's actual treatment regimen. For instance, if a patient is treated with a chemotherapeutic agent, the reference culture is treated with the same agent in the same concentration. The reference culture can be monitored genotypically and/or phenotypically to reflect actual progress of the proliferative disease or condition in the patient. Treatment of the reference culture need not be limited to anti-cancer therapies, but can reflect all of a patient's treatments. For instance, thrombolytic or anti-thrombogenic treatments can be applied to the reference culture to reflect a patient's treatment. Subcultures of either the prime culture or the reference culture can be used for further analysis, such as the genetic analysis techniques of the present invention. Preferably, since the reference culture is indicative of the current state in a patient of a proliferative disease or condition, subcultures of the reference culture are analyzed. At various points in the passage of the control culture and the reference culture, aliquots of cells from those cultures can be stored cryogenically or otherwise.

According to the present invention, abnormal proliferating cells prepared according to the culture methods of the present invention are then genetically analyzed for markers specific to the proliferative disease-state of the cells. The genetic data obtained thereby is used to simultaneously build and expand tandem genomic/proteomic cancer databases while coordinating present and future genomic/proteomic individual patient assays, thus providing a two-way feedback system. The cells that are analyzed typically are from subcultures of the prime or reference cultures. In this process nucleic acid is isolated from the cells and is analyzed to identify markers that are characteristic of abnormal proliferating cells. The isolated nucleic acid is DNA or RNA. The nucleic acid preferably is analyzed in a microarray for aberrant expression of one or more genes. Preferably, the microarray contains nucleic acids that are characteristic of known proliferative disease states, as well as nucleic acids, that are not corrected with known proliferative disease states so that previously unknown relationships between gene expression and a proliferative disease or condition may be identified. By the term “marker” it is meant any genotypic or phenotypic characteristic of a cell or cell population that, alone or in combination with other marker(s), can be used to identify the particular cell type. Markers can be, without limitation, genotypic, such as an insertion, deletion or substitution, or phenotypic, such as the presence of high levels of a receptor or a secreted peptide.

Methods for isolation of the nucleic acids of the cells are varied and typically differ from laboratory to laboratory. Further, certain analytical methods may require that the nucleic acid is prepared in a specific manner. Nucleic acid purification methods may be found in any one of a number of molecular biology laboratory texts, with purification products or systems also being commercially available from a variety of companies including Stratagene, Bio-Rad Laboratories and Amersham Pharmacia Biotech.

Methods for analysis of the nucleic acid derived from the cells also vary broadly. The presence of known proliferation markers, such as the aberrant expression of one or more genes such as, without limitation, the epidermal growth factor receptor (EGFR) cyclin D1, p16cyclin-kinase inhibitor, retinoblastoma (Rb), Tumor-derived Growth Factor B (TGFB) receptor/smad, MDM2 or p53 genes, may be determined by methodologies such as, without limitation, northern blotting or quantitative polymerase chain reaction (PCR) methods (i.e., RT-PCR).

Microarrays of either known DNAs or unknown DNAs, i.e., partially identified or unidentified expressed sequence tags (ESTs), are now commercially available from a number of commercial sources, such as Affymetrix, Incyte Pharmaceuticals, Stratagene, Nanogen and Rosetta Inpharmatics. The National Human Genome Research Institute (NHGRI) also has begun a collaborative research effort entitled “The Microarray Project,” which includes such efforts as the development of microarrays, robotic microarrayers and automated readers. DNA microarrays can include hundreds to many thousands of unique DNA samples covalently bound to a glass slide in a very small area. By hybridizing labeled RNA or mRNA to the array, the altered expression of one or more genes may be identified. In this embodiment of the present invention, total cellular RNA or mRNA (polyA⁺ mRNA) is isolated and labeled. Messenger RNA is labeled by synthesizing an oligo-dT-primed first DNA strand using reverse transcriptase and labeled deoxynucleotides such as, without limitation, Cy5-dUTP commercially available from Amersham Pharmacia Biotech. Radiolabeled nucleotides also can be used to prepare radioactive DNA probes. The labeled DNA, complementary to the mRNA, is hybridized to the microarray under sufficiently stringent conditions to ensure specificity of hybridization of the labeled DNA to the array DNA. The labeled array is then visualized. Visualization of the array may be conducted in a variety of ways. For instance, when the reading of the microarray is automated and the labeled DNA is labeled with a fluorescent nucleotide, the intensity of fluorescence for each discreet DNA of the microarray can be measured by an automated process using a confocal microscope or otherwise. The intensity of the fluorescence for each DNA sample in the microarray typically is directly proportional to the quantity of the corresponding species of mRNA in the cells from which the mRNA is isolated. It is possible to label cDNA from two cell types (i.e., normal and diseased proliferating cells) and hybridize equal amounts of both probes to a microarray to identify differences in RNA expression for both normal and diseased proliferating cells. Tools for automating microarray assays, such as robotic microarrayers and readers, are available commercially from companies, such as Nanogen, and are under development by the NHGRI. The automation of the microarray analytical process is desirable because of the huge number of samples that need to be deposited and interpreted.

DNA microarrays are possibly the more powerful tools to utilize in combination with the cell culturing method of the present invention due to the increased sensitivity of mRNA quantification protocols when a substantially pure population of proliferating cells are used. For their ease of use and their ability to generate large amounts of data, microarrays are preferred, when practicable. However, certain qualitative assays may be preferred to identify certain markers.

The presence of, or absence of, specific RNA or DNA species also may be identified by PCR procedures. Known genetic polymorphisms or translocations or insertions (i.e., retroviral insertions or the insertion of mobile elements) often can be identified by conducting PCR reactions with DNA isolated from cells cultured by the methods of the present invention. Where the sequence anomalies are located in exons, the genetic polymorphisms may be identified by conducting a PCR reaction using a cDNA template. Aberrant splicing of RNA precursors also may be identified by conducting a PCR reaction using a cDNA template.

Small or single nucleotide substitutions may be identified by the direct sequencing of the primary sequence of a given gene by the use of gene-specific oligonucleotides as sequencing primers. Single nucleotide mutations also may be identified through the use of allelic discrimination molecular beacon probes.

While the above-mentioned assays are useful in the analysis of nucleic acids derived from cells produced by the culture methods of the present invention, numerous additional methods are broadly known in the general field of molecular biology and molecular diagnostics that may be used in place of the above-referenced methods. These methods are considered to be within the scope of the present invention and along with the above-described methods are collectively included within the definition of “methods for characterizing the nucleic acid of proliferating cells,” as used herein and in the appended claims.

An important aspect of the present invention is to provide a system for conducting gene therapy protocols on various malignancies. For instance, chemotherapeutic treatments have been shown to be enhanced by transferring the Herpes Simplex Virus-Thymidine Kinase (HSV-TK) gene into cancer cells in order to create a suicide gene. Cancer cells carrying this mutated gene would be susceptible to the antiviral drug, ganciclovir (GCV). The Herpes Simplex Virus (HSV) produces a Thymidine Kinase that can convert GCV to ganciclovir-monophosphate (GCV-MP), a metabolite that is toxic to cells. Vectors can be produced that contain HSV1-TK from the human stereotype 1 virus as a suicide gene. Expression of HSV1-TK sensitizes transfected cells to GCV and also potentiates a strong bystander effect. The response of various malignancies to this gene therapy chemotherapy-sensitization protocol can be ascertained to provide important prognostic information.

An important further aspect of the present invention is to provide a system for screening specific tissue samples from individual patients for expressed cellular markers, secreted factors or antigens including tumor antigens characteristic of the tissue sample. A tissue sample from a patient is harvested and grown in a monolayer culture as described above. Culture medium in which the cultures or subcultures thereof is assayed for the presence or absence of certain factors, such as secreted tumor antigens such as, without limitation, PAI-1, u-PA, cancer associated serum antigen (CASA) or carcinoembryonic antigen (CEA). These factors may be detected through use of standard assays, such as radioimmunoassay (RIA) or enzyme-linked immunosorbent assay (ELISA), although other assays known to those skilled in the art may be used to detect and/or to quantify the soluble factors.

The cell cultures grown in this manner may also be assayed histochemically and/or immunohistochemically for identification or quantification of cellular or membrane-bound markers. Examples of such markers include, without limitation, CEA, tissue polypeptide specific antigen TPS, EGFR, TGFB receptor and mucin antigens, such as CA 15-3, CA 549, CA 27.29 and MCA. By screening tissue samples in this manner for production of such factors, markers or antigens, the cultured cells may be further identified aiding the physician in treatment strategies and as a prognosis indicator. Furthermore, by combining the use of the culture technique with assaying for markers as described herein, a treatment strategy for a disease state may be optimized and treatment progression may be monitored.

One important aspect of analyzing tissue culture medium is that complications of a proliferative disease state can be predicted. For instance, one common complication is thrombogenesis. A propensity towards blood clot formation can be detected in tissue culture medium by identifying thrombogenic or procoagulant factors such as, without limitation, cancer cell-derived coagulating activity −1 (CCA-1), the Lewis Y antigen (Ley), HLA-DR and other tumor procoagulants, such as cancer procoagulant (CP) and tissue factor (TF). By identifying production of thrombogenic factors, a physician can prescribe drug and/or exercise regimens, as appropriate, to prevent life and/or limb-threatening clotting.

Cells and/or tissue culture media from any of the prime culture, the reference culture or subcultures thereof can be analyzed for tumor aggressiveness and invasiveness markers. Presence of these markers, or absence thereof, is highly relevant to a patient's prognosis. Furthermore, the effect of a given therapy on any of these markers can be analyzed. For instance, a tumor may produce angiogenic factors such as, without limitation, vascular endothelial growth factor (VEGF), which would lead a doctor to give a patient a less favorable prognosis. Other markers can include, without limitation, factors which allow cancer cells to affix to organs other than those from which the cancer cells derive, for instance, beta-3 integrin, which participates in the ability of melanoma cells to adhere to blood vessel walls. However, the effectiveness of therapies can be assessed if the presence of the angiogenic marker is analyzed in segregated sites according to the method of the present invention. A physician can suppress a malignancy by preventing expression of factors or markers which cause the malignancy's aggressiveness or invasiveness.

One application of the present invention is the screening of chemotherapeutic agents and other anti-neoplastic therapies in tissue culture preparations of malignant cells from the patients from whom malignant samples are biopsied. Related anti-cancer therapies which also can be screened using the inventive system include radiation therapy and agents which enhance the cytotoxicity of radiation, as well as immunotherapeutic anti-cancer agents. Screening processes for treatments or therapeutic agents for non-malignant syndromes are also embraced within this invention and include, without limitation, agents which combat hyper-proliferative syndromes, such as psoriasis or wound healing agents. Nor is the present efficacy assay limited only to the screening of active agents which speed up (healing) or slow down (anti-cancer, anti-hyper-proliferative) cell growth because agents intended to enhance or to subdue intracellular biochemical functions may be tested in the present tissue culture system, also. For example, the formation or blocking of enzymes, neurotransmitters and other biochemicals may be screened with the present assay methods prior to treatment of the patient.

When a patient is to be treated for the presence of tumor, or other proliferative disease state, a tumor biopsy of >100 mg of non-necrotic, non-contaminated tissue sample is harvested from the patient by any suitable biopsy or surgical procedure known in the art and typically is placed in a shipping container for transfer to a laboratory in which the culturing of the cells according to the present invention is performed. Biopsy sample preparation generally proceeds as follows under a Laminar Flow Hood, which should be turned on at least 20 minutes before use. Reagent grade ethanol is used to wipe down the surface of the hood prior to beginning the sample preparation. The tissue sample is then removed under sterile conditions from the shipping container and is minced with sterile scissors. If the specimen arrives already minced, the individual tumor pieces typically are divided into four groups. Using sterile forceps, each undivided tissue sample quarter is then placed in 3 ml sterile growth medium (Standard F-10 medium containing 17% calf serum and a standard amount of penicillin and streptomycin) and systematically minced by using two sterile scalpels in a scissor-like motion or a mechanically equivalent manual or automated device having opposing incisor blades. This cross-cutting motion is important because the technique creates smooth cut edges on the resulting tumor multicellular particulates. Preferably, but not necessarily, the tumor particulates each measure 1 mm³. After each tissue sample quarter has been minced, one-half of the particulates are preserved in a cryoprotectant such as, without limitation, DMSO, glycerol or mannitol. The preserved particulates are aliquotted into small test tubes labeled with the patient's code, the date of explantation and any other distinguishing data, and then frozen in Dewar bottles containing liquid nitrogen at −40° C. The other half of the particulates are plated in culture flasks using sterile Pasteur pipettes (9 explants per T-25 or 20 particulates per T-75 flask) and each flask is labeled as described above. The explants should be evenly distributed across the bottom surface of the flask with initial inverted incubation in a 37° C. incubator for 5-10 minutes, followed by addition of about 5-10 ml sterile growth medium and further incubation in the normal, non-inverted position. Flasks are placed in a 35° C., non-CO₂ incubator. Flasks should be checked daily for growth and contamination. Over a period of a few weeks, with weekly removal and replacement of 5 ml of growth medium, the explants will foster growth of cells into a monolayer. With respect to the culturing of malignant cells, it is believed (without any intention of being bound by the theory) that by maintaining the abnormal proliferating cells within a multicellular particulate of the originating tissue, growth of the abnormal proliferating cells themselves is facilitated versus the overgrowth of fibroblasts (or other unwanted cells) which tends to occur when chemically suspended tumor cells are grown in culture.

The use of the above procedure to form a cell monolayer culture maximizes the growth of abnormal proliferating cells from the tissue sample and, thus, optimizes ensuing tissue culture assays. Enhanced growth of actual abnormal proliferating cells is only one aspect of the present invention; however, another important feature is the growth rate monitoring system used to oversee growth of the monolayer once formed. Once a primary culture and its derived secondary monolayer tissue culture has been initiated, the growth of the cells is monitored to ascertain the time to initiate the chemotherapy assay and to determine the growth rate of the cultured cells.

Monitoring of the growth of cells is conducted by counting the cells in the monolayer on a periodic basis, without killing or staining the cells and without removing any cells from the culture flask. The counting may be done visually or by automated methods, either with or without the use of estimating techniques known in the art (counting in a representative area of a grid multiplied by number of grid areas, for example). Data from periodic counting is then used to determine growth rates which may or may not be considered parallel to growth rates of the same cells in vivo in the patient. If growth rate cycles can be documented, for example, then dosing of certain active agents can be customized for the patient. The same growth rate can be used to evaluate radiation treatment periodicity, as well. It should be noted that with the growth rate determinations conducted while the monolayers grow in their flasks, the present method requires no hemocytometry, flow cytometry or use of microscope slides and staining, with all their concomitant labor and cost.

Protocols for monolayer growth rate generally use a phase-contrast inverted microscope to examine culture flasks incubated in a 37° C. (5% CO₂) incubator. When the flask is placed under the phase-contrast inverted microscope, ten fields (areas on a grid inherent to the flask) are examined using the 10× objective, with the proviso that the ten fields should be non-contiguous, or significantly removed from one another, so that the ten fields are a representative sampling of the whole flask. Percentage cell occupancy for each field examined is noted, and averaging of these percentages then provides an estimate of overall percent confluency in the cell culture. When patient samples have been divided between two or among three or more flasks, an average cell count for the total patient sample should be calculated. The calculated average percent confluency should be entered into a process log to enable compilation of data—and plotting of growth curves—over time. Monolayer cultures may be photographed to document cell morphology and culture growth patterns. The applicable formula is:

Percent confluency=estimate of the area occupied by cells/total area in an observed field.

As an example, therefore, if the estimate of area occupied by the cells is 30% and the total area of the field is 100%, percent confluency is 30/100, or 30%.

Adaptation of the above protocol, for non-tumor cells is straightforward and generally constitutes an equivalent procedure.

Dynamic assays, genomic/proteomic database building, and micro version genomic/proteomic patient assays, as well as active agent and/or radiation therapy screening using the cultured cells proceeds with subcultures of the prime culture or, preferably, of the reference culture. Cultured cells used for dynamic assay analyses are placed in incubation flasks. Therapy screening can be carried out in incubation flasks but generally proceeds using plates, such as microtiter plates. In a chemotherapy/radiotherapy assay, it is desirable to grow a control culture of a patient's cells in a culture parallel to the reference or prime culture. The control culture can be grown from skin cells of the patient, as an easy source of non-malignant cells, from the same organ from which the malignant cells are derived, or from other sources, so long as the cells are typical of non-malignant cells of the patient.

The performance of the chemosensitivity/radiosensitivity assay used for screening purposes depends on the ability to deliver a reproducible cell number to each row in a plate and/or a series of plates, as well as the ability to achieve an even distribution of cells throughout a given well. The following procedure assures that cells are reproducibly transferred from flask to microtiter plates, and cells are evenly distributed across the surface of each well.

The first step in preparing the microtiter plates is, of course, preparing and monitoring the monolayer as described above. The following protocol is exemplary and susceptible of variation as will be apparent to one skilled in the art. Cells are removed from the culture flask and a cell pellet is prepared by centrifugation. The cell pellet derived from the monolayer is then suspended in 5 ml of the growth medium and mixed in a conical tube with a vortex for 6-10 seconds. The tube is then rocked back and forth ten times. A 36 μl droplet from the center of the conical tube is pipetted onto one well of a 96-well plate. A fresh pipette is then used to pipette a 36 μl aliquot of trypan blue solution, which is added to the same well, and the two droplets are mixed with repeated pipette aspiration. The resulting admixture is then divided between two hemocytometer chambers for examination using a standard light microscope. Cells are counted in two out of four hemocytometer quadrants under 10× magnification. Only those cells which have not taken up the trypan blue dye are counted. This process is repeated for the second counting chamber. An average cell count per chamber is thus determined. Using means known in the art, the quadrant count values are checked, logged, multiplied by 10⁴ to give cells/ml, and the total amount of fluid (growth medium) necessary to suspend remaining cell aliquots is calculated accordingly.

After the desired concentration of cells in medium has been determined, additional cell aliquots from the monolayer are suspended in growth medium via vortex and rocking and loaded into a Terasaki dispenser known in the art. Cells to be used for dynamic assay analyses are plated onto culture flasks and placed in an incubator box at 37° C. under 5% CO₂. Aliquots of the remaining cell suspension are delivered into the microtiter plates using Terasaki dispenser techniques known in the art. A plurality of plates may be prepared from a single cell suspension as needed. Plates are then wrapped in sterile wet cotton gauze and incubated in an incubator box by means known in the art.

After the microtiter plates have been prepared, exposure of the cells therein to active agent and/or radiation is conducted according to the following exemplary protocol. During this portion of the inventive assay, the appropriate amount of specific active agent is transferred into the microtiter plates prepared as described above. A general protocol, which may be adapted, follows. Each microtiter plate is unwrapped from its wet cotton gauze sponge and microscopically examined for cell adhesion. Control solution is dispensed into delineated rows of wells within the grid in the microtiter plate, and appropriate aliquots of active agent to be tested are added to the remaining wells in the remaining rows. Ordinarily, sequentially increasing concentrations of the active agent or higher doses of radiation being tested are administered into progressively higher numbered rows in the plate. The plates are then rewrapped in their gauze and incubated in an incubator box at 37° C. under 5% CO₂. After a predefined exposure time, the plates are unwrapped, blotted with sterile gauze to remove the agent, washed with Hank's Balance Salt Solution, flooded with growth medium, and replaced in the incubator in an incubator box for a predefined time period after which the plates may be fixed and stained for evaluation.

Fixing and staining may be conducted according to a number of suitable procedures. The following is representative. After removal of the plates from the incubator box, culture medium is poured off and the plates are flooded with Hank's Balance Salt Solution. After repeated flooding (with agitation each time) the plates are then flooded with reagent grade ethanol for 2-5 minutes. The ethanol is then poured off. Staining is accomplished with approximately 5 ml of Giemsa stain per plate, although volume is not critical and flooding is the goal. Giemsa stain should be left in place 5 min. ±30 sec. as timing influences staining intensity. The Giemsa stain is then poured off and the plates are dipped three times in cold tap water in a beaker. The plates are then inverted, shaken vigorously, and air dried overnight (with plate lids off) on a rack on a laboratory bench. Cells per well are then counted manually or by automated and/or computerized means to derive data regarding chemosensitivity of cells at various concentrations of exposure. One particularly useful computer operating environment for counting cells is the commercially available OPTIMATE compiler, which is designed to permit an optical counting function well suited to computerized cell counting procedures and subsequent calculations.

The above procedures do not change appreciably when cell growth promoters are assayed rather than cell arresting agents, such as chemotherapeutic or radiotherapeutic agents. The present assay allows cell death or cell growth to be monitored with equal ease. In any case, optimization of use of the present system will involve the comparative testing of a variety of candidate active agents for selection of the best candidate for patient treatment based upon the in vitro results. One particularly advantageous embodiment of the above-described invention comprises a two-stage assay for cytotoxicity followed by evaluation of longer-term inhibitory effect. Chemotherapeutic agents may thus be evaluated separately for both their direct chemotherapeutic effect as well as for their longer duration efficacy.

As discussed in brief above, in parallel with growth of the prime or reference culture, a control culture can be grown. The control culture is a culture of normal cells taken from the same patient from whom the prime culture is collected. The control culture can derive from an epithelial cell sample or can be collected from the same organ as the prime culture so long as the control culture contains no abnormal proliferating cells. More than one control culture can be maintained. For instance, cultures of both normal skin cells and normal cells of an organ from which the malignancy is derived can be maintained. The value of maintaining a control culture is many fold. Primarily, the control culture serves as a negative control (or positive control depending upon the marker to be analyzed) in the various analyses to be carried out on the prime culture, the reference culture or subcultures thereof.

A second value of the control culture is an indicator of theratoxicity, the toxicity or undesirable effects of a given therapy upon normal cells. For instance, in the segregated analysis of chemotherapeutic agents described above, concomitant analysis of the same agents on segregated sites of the control culture would yield an indication of cytotoxicity of the agent with regard to malignant cells versus the theratoxicity of the agent to control cells. A therapeutic index can be calculated based on the ratio of cytotoxicity to malignant cells to theratoxicity. Cytotoxicity and theratoxicity can be quantified as a percentage or fraction of cells killed by a given therapy or as a percentage or fraction of cells surviving a given therapy. A therapeutic index is a ratio of these percentages or fractions and is reflective of the desirability of a given treatment in a patient. An optimal treatment would be maximally cytotoxic (or even cytostatic) to the malignant cells and minimally toxic to a patient's normal cells.

Other indices may be generated depending upon the desired effect of a therapy. For instance, if a desired therapy is designed to up-regulate a malignancy-specific antigen to promote destruction of the malignancy by a patient's immune system, an index could be generated to discern a treatment which reflects maximal up-regulation of the antigen in the malignant cells and minimal or negative up-regulation in a patient's normal cells. A similar index can be calculated based upon down-regulation of a desired marker (i.e., an angiogenic factor) which can be assayed as either a secreted or a cellular marker and reflects maximal down-regulation of the marker with minimal theratoxicity or other undesirable effects on the control culture.

Often the diseased cells express a cellular marker that is indicative of a certain disease state or lack thereof However, one aspect of the culture techniques of the present invention is that the cultured diseased cells do not necessarily have to be the cells expressing the factor to be assayed. One question that inevitably arises when considering whether a serum marker is indicative of a particular cancer cell is: which cells produce the marker, the cell or the tissue in which the cancer cells grow? By co-culturing the cancerous tissue within a multicellular particulate of its originating tissue, the cells (both the diseased cells or the surrounding cells) are better able to retain their production of characteristic markers.

Literally any active agent may be screened according to the present invention. Listing exemplary active agents is thus omitted here.

One important focus of the present invention thus includes the simplicity of the present system—cohesive multicellular particulates of the patient tissue to be tested are used to form cell monolayers; growth of those monolayers is monitored for accurate prediction of correlating growth of the same cells in vivo; differing concentrations of a number of active agents may be tested for the purpose of determining not only the most appropriate agent but the most appropriate concentration of that agent for actual patient exposure (according to the calculated cell growth rates), and the cultured cells can be tested for the presence of abnormal markers, such as genetic markers. It is also important to note in the context of the invention that the present system allows i vitro tests to be conducted in suspensions of tissue culture monolayers grown in nutrient medium, under fast conditions (a matter of weeks), rather than with single cell progeny produced by dilution cloning over long periods of time. In some cases, the present invention is a two-stage assay for both cytotoxicity and the longer-term growth inhibitory.

It is additionally possible to increase the value of the assay with the use of staining compositions and protocols designed to characterize the malignant cells thus grown. In other words, the tissue preparation and cell culturing technique itself offers a first assurance that the cells grown out of the tumor are really the malignant tumor cells and not fibroblasts or other non-malignant cells of no diagnostic value. As a separate confirmation, the present staining compositions and protocols offer a second, independent assurance that the cells subject to diagnostic or prognostic assay are, in fact, malignant cells in culture. One important characterization has to do with the nature of the malignant cells as epithelial, which is in turn an indicator of the carcinoma type of malignancy. Other characterizations of malignant cells are intended to fall within the scope of the present invention as well, although the characterization of the cells as epithelial or not is of primary importance.

The technique is practiced as follows. The same cell culturing and well distribution process is used as in the cytotoxicity assay described above, but rather than exposing the cells to chemotherapeutic or other agents, the cells are instead fixed and stained. With the stain or stain cocktail described below, the epithelial cells are identified by their intermediate filaments and/or specific membrane antigens by means of a monoclonal antibody immunoperoxidase technique. The fixative used can be any fixative which does not alter the cellular molecular markers of interest. The fixed stained cells are then counted. If the specimen is positive for epithelial cells, the process is complete. If the specimen is negative for epithelial cells, an independent fixing and staining process is subsequently completed, with fresh cells from identical wells, using Vimentin as a stain to confirm the non-epithelial nature of the cells.

The importance of having a stain or stain cocktail (i.e., antibody cocktail), as well as an overall protocol for identifying epithelial cells in biopsies of malignant tumors is as follows. In the basic cytotoxicity assay, the tissue culture technique is designed to grow out the cells of the tumor of origin and, in fact, consistently does so. Despite such reliable predictability, however, the fact that the cells of the tumor of origin did, in fact, grow out, and not fibroblasts or other cells, must be confirmed with independent proof before the cells can be used with complete assurance in the appropriate patient assay(s). The present technology provides a means to obtain this confirmation which, in turn, furthers the interests of good laboratory and medical practice.

As a general consideration, the staining compounds or compositions of interest for use in the present technology are those which bind with cellular molecular markers unique either to epithelial or to non-epithelial cells. A further aspect of the invention, therefore, inheres in the following two aspects: the improvement of the cytotoxicity assay by adding the epithelial staining protocol with any known epithelial stain; and the further improvement wherein specially designed stain cocktails maximize the likelihood that the presence of any known intermediate filament or specific membrane antigen characteristic of epithelial cells will be identified, if present.

Many carcinomas are positive for any one of the intermediate filaments or specific membrane antigens characteristic of epithelial cells. Virtually all, if not all, carcinomas are positive for one of a number of such intermediate filaments or specific membrane antigens. For example, “epithelial membrane antigen” (EMA) glycoproteins are known in the art and can be bound with various anti-epithelial membrane antigen antibodies including monoclonal antibodies. Cytokeratin is another important epithelial cell marker and binding reagents including monoclonal antibodies are available which are specific to cytokeratin. While antisera can be raised in vivo against markers such as EMA glycoproteins and cytokeratin, as a practical matter, commercially available polygonal or monoclonal antibodies are used in the following protocols, with monoclonal antibodies being preferred.

Binding of the epithelial marker is revealed with associated staining procedures and reactions which give a visual indication that the marker binding took place. Those skilled in the art appreciate various techniques already available—in the general field of “immunocytochemistry”—to reveal antibody-antigen reactions.

One known way to accomplish this visualization when antibody binding reagents are used is with the “labeled streptavidin procedure.” In this procedure, after the specimen is exposed to antibodies specific to the target antigen, a secondary “link” antibody is added. The secondary biotinylated “link” antibody consists of anti-mouse and anti-rabbit antibodies which bind universally to most primary monoclonal or polyclonal antibodies. The “link” will also connect to the tertiary reagent (peroxidase-labeled streptavidin) through chemical bonding between the biotin on the secondary reagent and the streptavidin on the streptavidin/peroxidase conjugate. Staining is completed by incubating the specimen and primary, secondary and tertiary agents in the presence of a chromogen, so that the peroxidase and the chromogen form a visible precipitate. Alternatively, a fluorescein-based detection system can be used to visualize the primary antibody, or a third alternative known in the art as the digoxigenin-conjugated detection system may be used.

Of the various epithelial markers, three have received the most widespread attention in the literature: EMA glycoproteins, cytokeratin, and carcinoembryonic antigen. In the context of this invention, the first two are the most important because literally any epithelial cell will have at least either one EMA glycoprotein on the surface thereof or a cytokeratin intermediate filament present. Therefore, the present invention resides not only in binding and staining for an epithelial marker on the surfaces of the specimen cells, but in simultaneously assaying for either or both of EMA glycoprotein(s) and cytokeratin. The cocktails of the present invention, therefore, contain binding reagents for both EMA glycoproteins and cytokeratin and, importantly, are selected to include the most generally applicable binding reagents in combination so that the cocktail has the broadest binding scope possible. The cocktails identified in Examples 1 and 2, for example, represent a combination of two general binding reagents (containing a total of three monoclonal antibodies) for cytokeratin, admixed with a general binding reagent for EMA glycoprotein. The dual benefit of this admixture of general binding agents is that the incidence of false negatives for epithelial cells is minimized and the visible staining reactions are generally stronger when the combined binding reagents are used in lieu of a single binding reagent.

Although the binding reagents and other reagents identified in the Examples are the preferred reagents for use in the context of the invention, the invention is intended to encompass epithelial-specific binding and staining reagents generally. These include, without limitation, Boehringer-Mannheim AE1 anti-cytokeratin antibody; Boehringer-Mannheim AE3 anti-cytokeratin antibody; Boehringer-Mannheim AE1/AE3 anti-cytokeratin antibody (AE1 and AE3 in admixture); Becton-Dickinson CAM 5.2 antibody; DAKO EMA antibody; Biomeda's Anti-Cytokeratin Cocktail CK22; Biomeda's Anti-Cytokeratin Cocktail CK23; Biomeda's Anti-Pan-Cytokeratin CK56; Biomeda's polyclonal goat or rabbit anti-cytokeratin antisera; ScyTek Laboratories anti-EMA antigen antibody clone E29; and many others. Those skilled in the art and in possession of the guidance provided herein can readily determine alternative, equivalent binding and staining reagents and cocktails to accomplish the disclosed result. These binding agents and cocktails may be used in combination with any known visualization system, such as the streptavidin-, fluorescein- and digoxigenin-conjugated systems identified above.

As a control, Vimentin antibody is used as a binding alternative either in conjunction with binding and staining of the test cells or subsequently thereto. In the context of this invention, Vimentin can be considered a binding reagent which is specific to non-epithelial cells.

In a further aspect of the present invention, immunological markers may be monitored in applications requiring up- or down-regulation of such markers (i.e., major histocompatability complex molecules). This aspect of the present invention can be especially useful in transplantation applications where, for instance, through chemical or biological means, rejection of transplanted cells is sought to be avoided by down-regulation of the various transplantation antigens present on the cells to be transplanted. The present invention would be especially useful in monitoring such immunoregulation.

Lastly, cell morphology can be assayed by culturing cells of, i.e., the prime culture or the reference culture; removing the cells from the surface upon which they grow; centrifuging the cells into a loose pellet; and growing the cell pellet over a defined time period. By growing cells in this manner, it is possible to view the cohesive morphology of cells in a cluster resembling a tumor.

### EXAMPLE 1

### Radiation Therapy

Separate 50 mg samples from residual tissue from specimens of three human glioblastomas and one human ovarian carcinoma were minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. Each 50 mg sample was minced and was divided into four groups of particulates and each of 16 groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the 16 flasks. Each cell pellet was then suspended in 5 ml of the above-described medium and was mixed in a conical tube with a vortex for 6-10 seconds, followed by manual rocking back and forth ten times. A 36 ml droplet from the center of each tube was then pipetted into one well of a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and by means known in the art, the optimum concentration of cells in the medium was determined.

Accommodating the above calculations, additional cell aliquots from the 16 monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours later, the cells were irradiated using a Siemens Stabilipan X-ray machine at 250 kVp, 15 mA with a dose rate of 75 rad/minute. For each radiation dose from 1 Gy to 6 Gy, cell number per well was monitored as a function of time through five days post-irradiation.

Cell number relative to controls was determined and survival curves were fit to the data. The rate of decrease in survival as a function of time was proportional to dose. A differential radiation response among the four cell lines was observed.

### EXAMPLE 2

### Immunotherapy

Separate 50 mg samples from residual tissue from specimens of a human brain tumor, renal carcinoma, and breast carcinoma were minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. Each 50 mg sample was minced and was divided into four groups of particulates and each of 12 groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the twelve flasks. Each cell pellet was then suspended in 5 ml of the above-described medium and was mixed in a conical tube with a vortex for 6-10 seconds, followed by manual rocking back and forth ten times. A 36 ml droplet from the center of each tube was then pipetted into one well a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and by means known in the art, the optimum concentration of cells in the medium was determined.

Accommodating the above calculations, additional cell aliquots from the 12 monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours post-plating, the Activated Natural Killer (ANK) cells were delivered into a row of six wells by means of a micropipette. In each microtiter plate, three rows of six wells each served as controls. The effector (ANK cells) target cell (tumor cells) ratio varied from 2.5:1 to 20:1. The ANK cells were exposed to the target cells for four hours. Subsequently, the wells were washed with Hank's Balanced Salt Solution and the number of ANK cells remaining in the wells was observed with a phase contrast microscope. This process was repeated until no ANK cells remained in the wells (usually three washes). Following removal of the ANK cells, the tumor cells were incubated in the wells for another 24 hours.

Cell number relative to control was determined. For the three tumor types increasing the effector target cell ratio from 2.5:1 to 20:1 resulted in an increase in the number of tumor cells killed by the ANK cells.

### EXAMPLE 3

### Gene Therapy/Antisense Oligonucleotides

A 50 mg sample from a residual human mesothelioma was minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. The 50 mg sample was minced and was divided into four groups of particulates and each of four groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the four flasks. Each cell pellet was then suspended in 5 ml of the above-described medium and was mixed in a conical tube with a vortex for 6-10 seconds, followed by manual rocking back and forth ten times. A 36 ml droplet from the center of each tube was then pipetted into one well of a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and by means known in the art, the optimum concentration of cells in the medium was determined.

Accommodating the above calculations, additional cell aliquots from the four monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours post-plating, the antisense oligonucleotide for the urokinase-type plasminogen activator receptor (uPAR) was delivered to wells in the microtiter plate. Proteolysis of plasminogen to plasmin by urokinase-type plasminogen activator has been implicated in the processes of tumor cell proliferation and invasion. The concentrations of the uPAR antisense oligonucleotide were 1, 10 and 100 micromolar. uPAR sense and missense oligonucleotides at the concentrations of 1, 10 and 100 micromolar served as controls. The tumor cells were exposed to the oligonucleotides for 24 hours and then the agents were removed. The cells were allowed to incubate for another 72 hours so that inhibition of cell proliferation could be observed.

Cell number relative to control was then determined. Antisense oligonucleotides to uPAR suppressed the proliferative activity of the tumor cells in a concentration-dependent manner.

### EXAMPLE 4

### Gene Therapy/HSV-TK Ganciclovir Suicide Gene

A 50 mg sample from a residual human ovarian cancer was minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. After each tissue sample quarter has been minced, one-half of the particulates was preserved in DMSO. The preserved particulates were aliquotted into small test tubes labeled with the patient's code, the date of explantation and any other distinguishing data, and frozen in Dewar bottles containing liquid nitrogen at −40° C. The other half of the particulates was plated in culture flasks using sterile Pasteur pipettes (9 explants per T-25 or 20 particulates per T-75 flask) and each flask was labeled as described above. Visual confirmation was made that the particulates were evenly distributed along the bottom of each flask and the flasks were placed in a 35° C. incubator. Over a period of a few weeks, with weekly removal and replacement of 5 ml of growth medium, the particulates grew into monolayers.

Enough cells were then removed from the monolayers grown in the flasks for centrifugation into standard size cell pellets. Each cell pellet was then carefully removed from the centrifuge tube and plated onto a sterile culture flask in Standard F-10 medium containing 17% calf serum and a standard amount of penicillin and streptomycin and placed in a 37° C. CO₂ incubator.

Twenty-four hours post-plating, the control group, consisting of one-half of the tumor pellets, received by needle transfer an adenovirus vector carrying the lacZ marker gene and an appropriate dose of GCV. The treatment group received by needle transfer an adenovirus vector carrying the HSV-TK gene and an appropriate dose of GCV. The pellets were allowed to incubate for another 72 hours.

HSV-TK/GCV gene therapy produced moderate and diffuse degeneration of the cancer cells in the treatment group when compared to the control group.

### EXAMPLE 5

### Combination Chemotherapy

Separate 50 mg samples from residual tissue from specimens from four human ovarian tumors were minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. Each 50 mg sample was minced and divided into four groups of particulates and each of 16 groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the 16 flasks. Each cell pellet was then suspended in 5 ml of the above-described medium and was mixed in a conical tube with a vortex for 6-10 seconds, followed by manual rocking back and forth ten times. A 36 ml droplet from the center of each tube was then pipetted into one well of a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and, by means known in the art, the optimum concentration of cells in the medium was determined.

Accommodating the above calculations, additional cell aliquots from the 16 monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours post-plating, the chemotherapeutic agent Taxol was applied to the wells in the microtiter plates. The first three treatment rows in the plates (rows 2, 3, and 4) were designed to have escalating Taxol doses (1.0, 5.0, and 25 μM) with a fixed carboplatin dose (200 μM). The last three treatment rows in the plates (rows 6, 7, and 9) were designed to have a fixed Taxol dose (5 μM) with an escalating carboplatin dose (50, 200, and 1000 μM). Rows 5 and 9 served as a control. The Taxol exposure time was two hours. Twenty-four hours later, the cells in the wells were exposed to carboplatin for two hours. The tumor cells in the wells were then incubated for another 48 hours.

Cell number relative to control was determined. For the cells from the four tumor specimens, a dose response relationship was observed for both the escalating Taxol/fixed carboplatin and fixed Taxol/escalating carboplatin treatment schema.

### EXAMPLE 6

### Hormonal Therapy

Separate 50 mg samples from residual tissue from specimens from four human breast tumors were minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. Each 50 mg sample was minced and divided into four groups of particulates and each of 16 groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the 16 flasks. Each cell pellet was then suspended in 5 ml of the above-described medium and mixed in a conical tube with a vortex for 6-10 seconds, followed by manual rocking back and forth ten times. A 36 ml droplet from the center of each tube was then pipetted into one well of a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and by means known in the art, the optimum concentration of cells in the medium was determined.

Accommodating the above calculations, additional cell aliquots from the 16 monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours post-plating, the antiestrogenic compound Tamoxifen was delivered to wells in the microtiter plates. A stock solution of Tamoxifen was initially prepared by dissolving 1.5 mg of Tamoxifen powder in 1 ml of absolute ethanol and then adding 9 ml of growth medium. This stock solution was then used to make Tamoxifen solutions in the concentration range of 10 nM to 20 μM. Six doses of Tamoxifen were used for cells from each of the four breast tumor specimens. An ethanol solution at a concentration equivalent to that at the highest Tamoxifen concentration served as a control. The tumor cells were exposed to Tamoxifen for 24 hours and then the agent was removed. The cells were allowed to incubate for another 72 hours so that inhibition of cell proliferation could be observed.

Cell number relative to control was then determined. There was no effect observed when the ethanol-only control wells were compared to the growth medium-only control wells. The cells of two of the four breast specimens tested showed an inhibition of cell proliferation by Tamoxifen exposure. These responses occurred in the mid-to-high Tamoxifen concentration ranges.

### EXAMPLE 7

### Differentiating Agent Therapy

### Biological Response Modification

Separate 50 mg samples from residual tissue from specimens from four human breast tumors were minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. Each 50 mg sample was minced and divided into four groups of particulates and each of 16 groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the 16 flasks. Each cell pellet was then suspended in 5 ml of the above-described medium and mixed in a conical tube with a vortex for 6-10 seconds, followed by manual rocking back and forth ten times. A 36 ml droplet from the center of each tube was then pipetted into one well of a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and by means known in the art, the optimum concentration of cells in the medium was determined.

Accommodating the above calculations, additional cell aliquots from the 16 monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours post-plating, the differentiating agent retinoic acid was delivered to wells in the microtiter plates. A stock solution of retinoic acid was initially prepared by dissolving retinoic acid powder in 1 ml of DMSO and then adding 9 ml of growth medium. This stock solution was then used to make retinoic acid solutions in the concentration range of 0.1 to 1.0 mM. Six doses of retinoic acid were used for cells from each of the four breast tumor specimens. A DMSO solution at a concentration equivalent to that at the highest retinoic acid concentration served as a control. The tumor cells were exposed to retinoic acid for 24 hours and then the agent was removed. The cells were allowed to incubate for another 72 hours so that inhibition of cell proliferation could be observed.

Cell number relative to control was then determined. There was no effect observed when the DMSO-only control wells were compared to the growth medium-only control wells. The cells of three of the four breast specimens tested showed an inhibition of cell proliferation by retinoic acid exposure. These responses occurred in the mid-to-high retinoic acid concentration ranges.

### EXAMPLE 8

### Combined Modality Therapy Drug/Radiation

Separate 50 mg samples from residual tissue from specimens from two human brain tumors and two human ovarian tumors were minced in medium with sterile scissors to a particle size of roughly 1 mm³ and with a particle size distribution between about 0.25 and about 1.5 mm³. The medium was Standard F-10 medium containing 17% calf serum and a standard amount of penicillin and streptomycin. Each 50 mg sample was minced and divided into four groups of particulates and each of 16 groups was charged to a separate labeled culture flask containing the above-described medium. Visual confirmation was made that the particulates were evenly distributed along the bottom of each flask and the flasks were placed in a 35° C., non-CO₂ incubator. Flasks were checked daily for growth and contamination. Over a period of a few weeks, with weekly removal and replacement of 5 ml of growth medium, the particulates grew into monolayers.

Enough cells were then removed from the monolayers grown in the flasks for centrifugation into standard size cell pellets for each of the 16 flasks. Each cell pellet was then suspended in 5 ml of the above-described medium and mixed in a conical tube with a vortex for 6-10 seconds, followed by manual rocking back and forth ten times. A 36 ml droplet from the center of each tube was then pipetted into one well of a 96-well microtiter plate together with an equal amount of trypan blue, plus stirring. The resulting admixture was then divided between two hemocytometer quadrants for examination using a standard light microscope. Cells were counted in two out of four hemocytometer quadrants under 10× magnification—only those cells which had not taken up the trypan blue dye were counted. This process was repeated for the second counting chamber. An average cell count per chamber was calculated and by means known in the art, the optimum concentration of cells in the medium was determined.

Accommodating the above calculations, additional cell aliquots from the 16 monolayers were separately suspended in growth medium via vortex and rocking and were loaded into a Terasaki dispenser adapted to a 60-well plate. Aliquots of the prepared cell suspension were delivered into the microtiter plates using Terasaki dispenser techniques known in the art. Cells were plated into 60-well microtiter plates at a concentration of 100 cells per well.

Twenty-four hours post-plating, cells in the microtiter plate wells were exposed to the chemotherapeutic agent Taxol. One set of plates was designed to have escalating Taxol doses with (0.5-25.0 μM) with a fixed radiation dose (2 Gy). A second set of plates was designed to have a fixed Taxol dose (5 μM) with an escalating radiation dose (1 Gy-6 Gy). The cells in the plates were irradiated using a Siemans Stabilipan X-ray machine operating at 250 kVp, 15 mA with a dose rate of 75 rad/min.

For each of the two treatment schema, cell number per well was monitored as a function of time through five days post-treatment. Cell number relative to controls was determined and survival curves were fit. A differential response among the cells from the four tumor specimens was observed. Both additive and synergistic cell killing were noted.

### EXAMPLE 9

### Initiation of a Prime Culture

A tumor biopsy of approximately 100 mg of non-necrotic non-contaminated tissue was harvested from the patient by surgical biopsy and transferred to the laboratory in a standard shipping container. Biopsy sample preparation proceeded as follows: Reagent grade ethanol was used to wipe down the surface of a Laminar Flow Hood. The tumor was then removed under sterile conditions from its shipping container and cut into quarters with a sterile scalpel. Using sterile forceps, each undivided tissue quarter was then placed in 3 ml sterile growth medium (Standard F-10 medium containing 17% calf serum and a standard amount of penicillin and streptomycin) and was systematically minced by using two sterile scalpels in a scissor-like motion. The tumor particulates each measured about 1 mm³. After each tumor quarter was minced, the particulates were placed in culture flasks using sterile Pasteur pipettes (9 explants per T-25 or 20 particulates per T-75 flask). Each flask was then labeled with the patient's code, the date of explantation and any other distinguishing data. The explants were evenly distributed across the bottom surface of the flask, with initial inverted incubation in a 37° C. incubator for 5-10 minutes, followed by addition of about 5-10 ml sterile growth medium and further incubation in the normal, non-inverted position. Flasks were placed in a 35° C., non-CO₂ incubator. Flasks were checked daily for growth and contamination. Over a period of a few weeks, with weekly removal and replacement of 5 ml of growth medium, the explants grew out into a monolayer.

### EXAMPLE 10

### Unified Tracking System

### A. Growth Rate

Following initiation of prime cell culture of a tumor specimen, the growth rate of the cells was determined until the chemosensitivity assay was performed. During this time period, the growth was monitored by observing the percent of confluency of the cells in a flask. These data provide information valuable as a correlation to possible growth of the tumor in the patient, as well as for the interpretation of the results of the chemosensitivity assay.

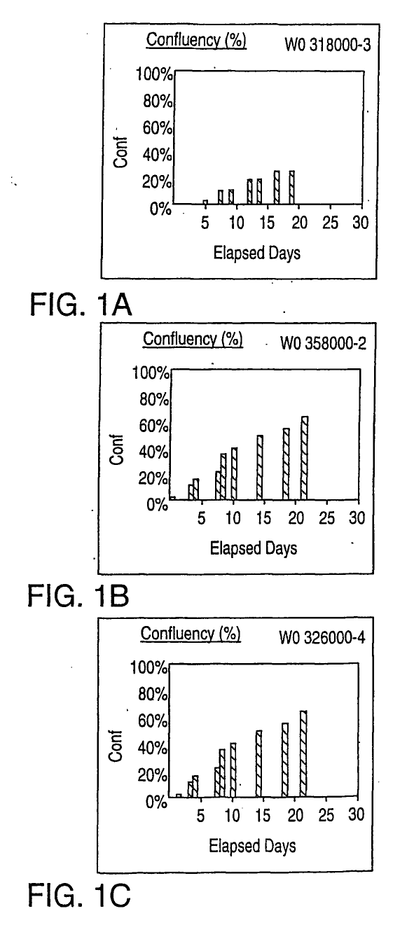

Three examples of growth rate data are shown in FIGS. 1A-1C. The percent of confluency of the cultured cells is plotted as a function of time after the initial seeding of the tissue specimen.

| | |
|---|---|
| Slow Growth Rate (FIG. 1A): | 25% confluent after 19 days |
| Moderate Growth Rate (FIG. 1B): | 60% confluent after 21 days |
| Fast Growth Rate (FIG. 1C): | 90% confluent after 21 days |

### B. Immunohistochemical Staining for Cell Characterization, etc.

Many tumor specimens will contain a mixture of cancer and normal cells. Although in many cases tumor cells will readily grow in tissue culture while the normal cells will not, it is important to be able to distinguish the two cell types. Using immunoperoxidase techniques to stain cells for various intermediate filaments, the differences between normal (fibroblast-like) cells and cells from epithelial tumors were characterized. These techniques can also be used to identify other tumor cell characteristics which may have prognostic value.

An initial attempt at cultured cell characterization has been known to use known epithelial tumor cell lines and a known fibroblast cell line. The epithelial tumor cell lines all have stained positively for a mixture (“cocktail”) of epithelial intermediate filament antibodies (not every line, however, has stained positively for the three antibodies within the mixture [AE1/AE3; Cam 5.2; EMA]). Some of the epithelial tumor cells in culture also stained mildly positive for an antibody against an intermediate filament characteristic of fibroblasts (vimentin). When staining for fibroblast intermediate filament (vimentin) in cell culture, all fibroblast cells were positive. Some focal staining by epithelial tumor cells for vimentin was also present.

| | Epithelial Cocktail | Vimentin |
|---|---|---|
| Epithelial tumor cells | ++ | + |
| Fibroblasts | − | ++ |

Testing of intermediate filaments with antibodies for epithelial cells and Vimentin appears to be a method of distinguishing certain characteristics of tumor and normal cells.

### C. Response to Chemotherapy

The tissue culture chemosensitivity assay has been refined to make it more sensitive for the detection of damage produced by a variety of chemotherapeutic agents. The initial alteration was to allow a 24-hour time period between plating of cells in microtiter wells and the exposure to drugs. This time interval permits cells to be in an active state of proliferation where they are more sensitive to cell cycle active agents. The second change was to initiate a long-term assay (growth inhibition assay) over a period of about 72 hours. The short-term assay is conducted 24-72 hours after the therapeutic agent is added. The longer time between drug exposure and assay allows for the detection of cell damage which occurs over a protracted period and requires several cell division cycles before it becomes apparent. “CI” (Cytotoxicity Index) is a measure of the relative survival rates of a given cell culture. It is calculated according to the formula:

$$CI = \frac{( 1 \text{-} No . of cells in treated wells )}{No . of cells in control wells}$$

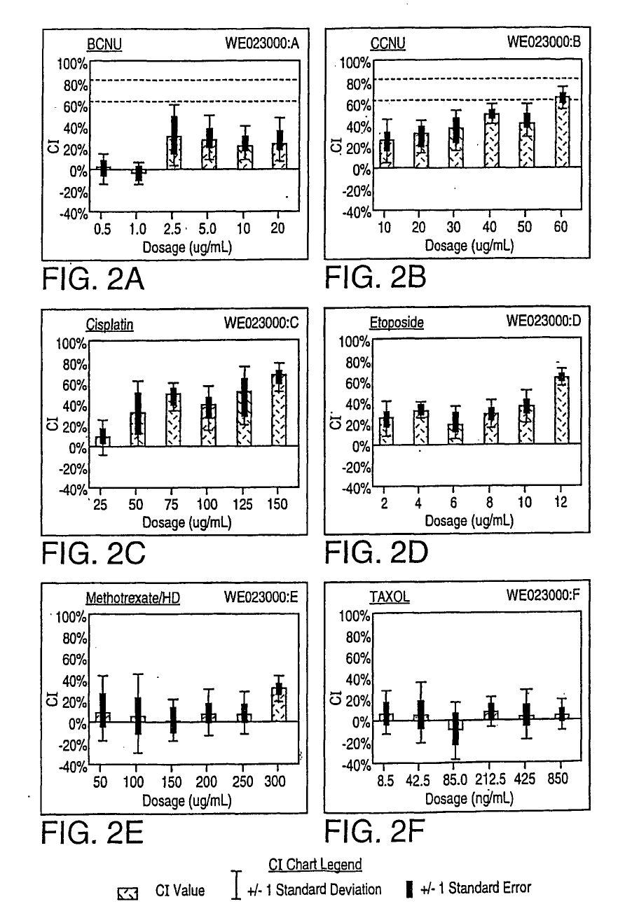

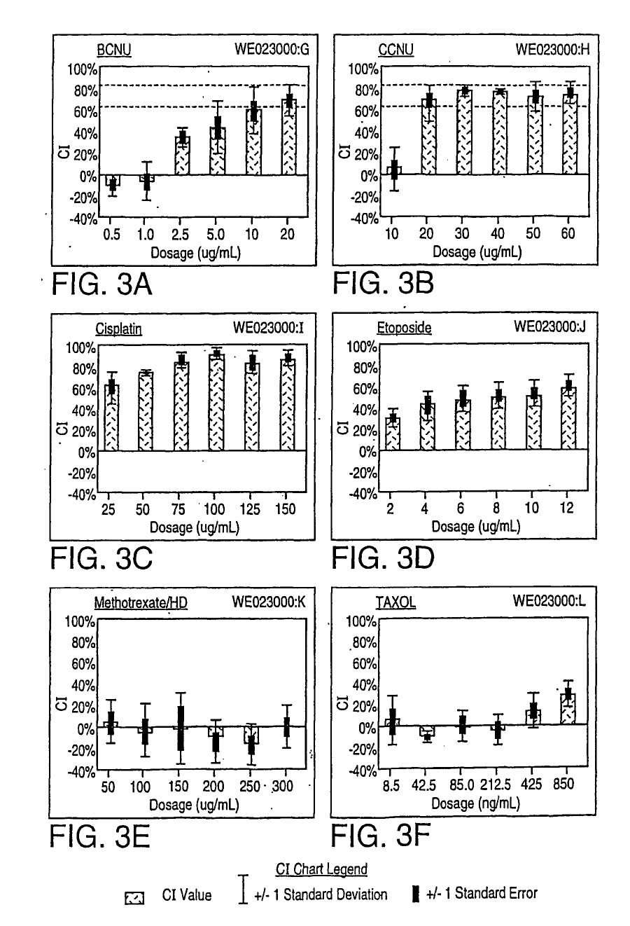

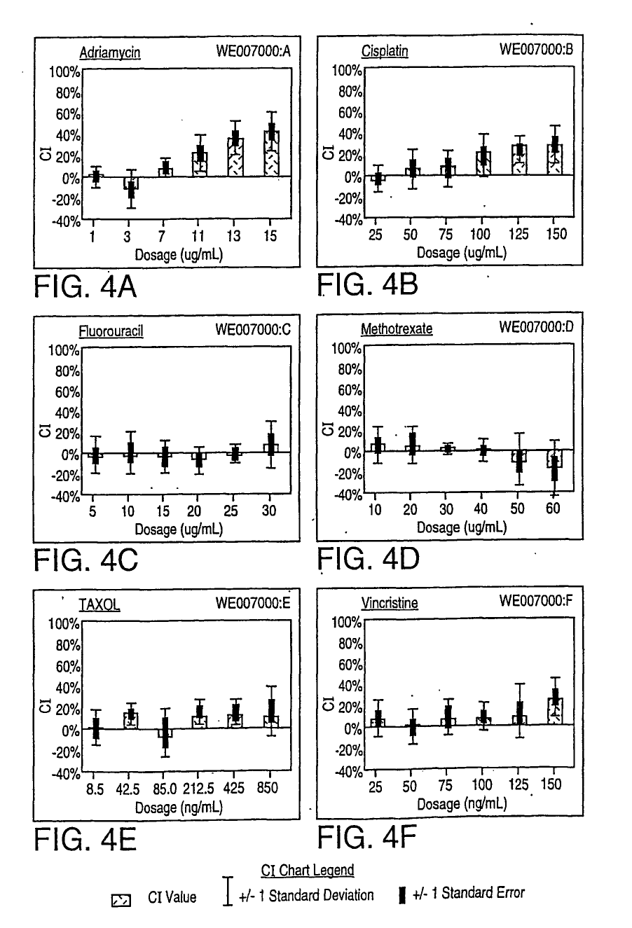

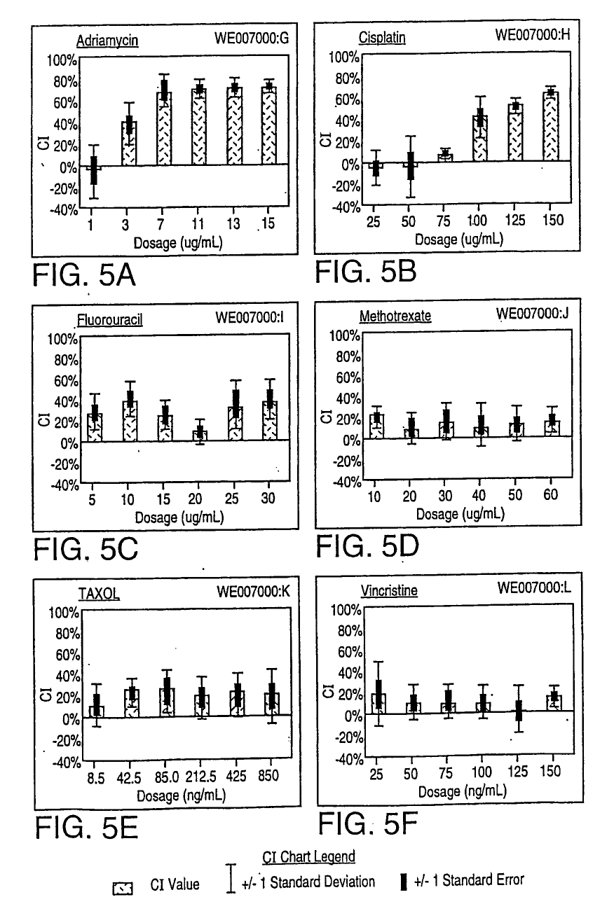

The data for a short-term assay and a long-term assay performed on two sets of patient cultured cells are presented in FIGS. 2A-2F through FIGS. 5A-5F. The long-term assay (FIGS. 3A-3F and FIGS. 5A-5F) may both accentuate a positive result obtained from the short-term assay (FIGS. 2A-2F and FIGS. 4A-4F) and reveal an effect not observed during the short-term assay. The long-term assay is now incorporated into the tissue culture chemosensitivity on a routine basis.

### D. Response to Radiation Therapy

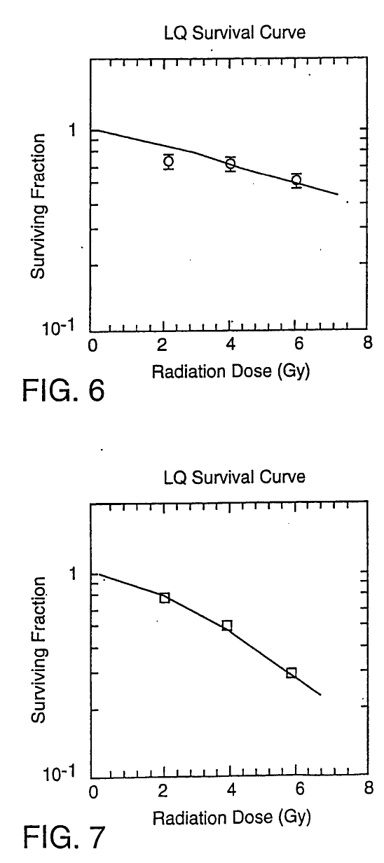

The use of the microtiter well assay to analyze the direct effect of radiation therapy on tumor cells in culture has resulted in a rapid evaluation method for the determination of inherent cellular radiation response. As an example, two radiation dose-response curves generated from the microtiter well assay are presented in FIGS. 6 and 7. The cells from the tumor specimen in FIG. 6 are more resistant than those of the specimen in FIG. 7. The more resistant tumor has been previously irradiated.

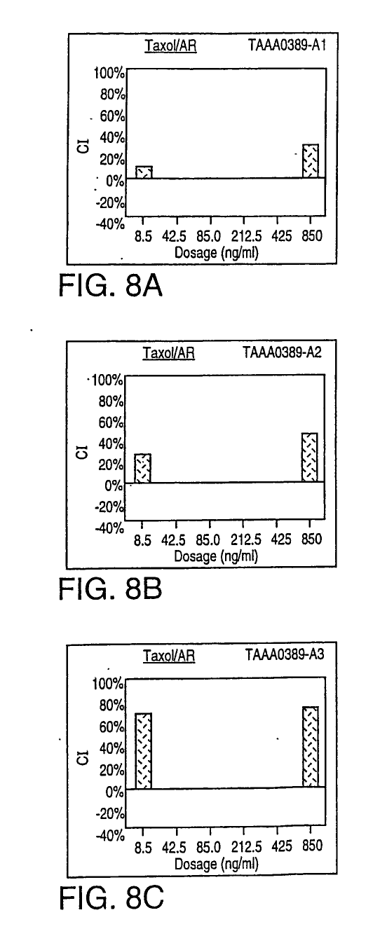

The microtiter well assay is ideally suited for examination of the interaction of chemotherapeutic agents and radiation. Issues such as the differential sensitivity of drug/radiation combinations and the timing of drug/radiation combinations may be directly addressed with this system. An illustration of chemotherapeutic agent enhancement of radiation response is presented in FIGS. 8A-8C.

| | |
|---|---|
| FIG. 8A: | Radiation-only at 2 Gy and 4 Gy |
| FIG. 8B: | Taxol 8.5 ng/ml + 2 Gy and 4 Gy |
| FIG. 8C: | Taxol 42.5 ng/ml + 2 Gy and 4 Gy |

### E. Response to Cellular Immunotherapy

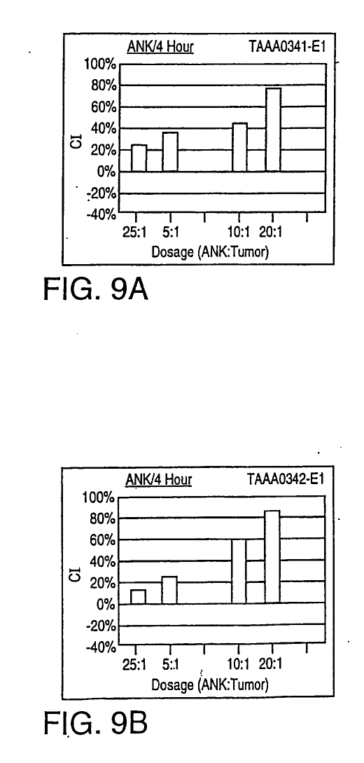

Activated lymphocytes are being used as a treatment for some types of cancer. These Activated Natural Killers (ANK) cells have been shown to mediate highly efficient cell killing for some tumor types. The microtiter well assay can be utilized to make a rapid assessment of ANK-induced tumor target cell killing. An illustration of two such interactions is presented in FIGS. 9A and 9B.

In FIGS. 9A and 9B, the target cells were from a melanoma and a renal carcinoma, respectively. The target cells were exposed to the ANK cells for four hours and then the assay was performed. The effector target cell ratio varied from 1:2.5 to 1:20. The data show increasing cell killing as a function of increasing effector target ratio.

### F. Use of Tissue Culture Medium for Determination of Factors with Possible Prognostic/Biological Significance

A number of substances secreted by tumor cells, such as tumor associated antigens and plasminogen activators and inhibitors, are believed to regulate a variety of processes involved in the progression of malignant disease. Many of these factors are produced by tumor cells growing in tissue culture and are secreted into the growth medium. The measurement of these factors in the medium from cell cultures of tumor specimens may prove to be of predictive value in the assessment of the biological behavior of individual cancers.

Preliminary work in this area has been on the detection of plasminogen activator inhibitor in the growth medium of glioblastoma cell lines. Plasminogen activator inhibitor expression has been shown to be increased in patients with malignant brain tumors. Medium from glioblastoma cell lines showed an increase in plasminogen activator inhibitor when compared to the medium alone.

Any or all of the steps of the unified assays and culturing techniques of the present invention may be automated. Indices can be automatically calculated by a computer which is programmed appropriately. Data can be input into the computer either manually or automatically, into a spreadsheet or database program, or the like. The spreadsheet or database program can be programmed to reduce the data to the indices described above, or to any other relevant form, i.e., graphical or figurative representations of the data.

In one example, the cells to be assayed are grown on microtiter plates and assayed for their sensitivity to a chemotherapeutic agent according to the above-described protocols. The microtiter plates are read on an optical scanner and data from the scanner is automatically exported to a computer for calculation of a therapeutic index. Other types of scanners may be utilized depending upon the assay. For instance, a scanner for reading RIA data would be provided if the assay is an RIA assay.

### EXAMPLE 11

### Characterization of Nucleic Acids of a Tumor Specimen

A tumor biopsy was obtained and cultured according to the culture method of the present invention and total cellular RNA was isolated. PolyA⁺ RNA was isolated from the total cellular RNA by passage of the total cellular RNA over an oligo-dT cellulose column. Approximately 1 μg of polyA⁺RNA was labeled with Cy3-dUTP, commercially available from Amersham Pharmacia Biotech, in a first strand cDNA synthesis reaction using reverse transcriptase. The fluorescently labeled cDNA was hybridized under stringent conditions to a microarray of DNA corresponding to genes expressed in commercially available cancer cells. The intensity of the fluorescence for each DNA of the microarray was measured and stored in a computer database.

The present invention has been described with reference to specific details of particular embodiments thereof. It is not intended that such details be regarded as limitations upon the scope of the invention except insofar as and to the extent that they are included in the accompanying claims.

We claim:

1. A method for preparing genomics/proteomics cancer databases comprising the steps of:
a. culturing proliferating cells by the steps of:
i. collecting a tissue sample including the proliferating cells;
ii. mechanically dividing the sample into cohesive multicellular particulates; and
iii. growing a tissue culture monolayer from the multicellular particulates;
b. purifying a nucleic acid sample from cells of the monolayer;
c. analyzing the nucleic acid using a method for characterizing the nucleic acid of proliferating cells, thereby generating genetic data specific to the tissue sample;
d. treating the nucleic acid sample to a drug wherein a specific gene-drug response is elicited;
e. collecting clinical data specific to the patient from whom the tissue sample was collected;
f. gathering the genetic data and the clinical data to form a data set; and
g. correlating the genetic data and the clinical data to create a gene relationship database structure.

2. The method of claim 1, further comprising the step of performing genomic, proteomic, and a combination of genomic/proteomic dynamic assays on the tissue culture monolayer.

3. The method of claim 2, further comprising the step of using an ATP bioluminescence assay to determine apoptosis levels of the proliferating cells.

4. The method of claim 3, further comprising dividing the monolayer into two halves, wherein one-half of the cells of the monolayer are unfrozen and the other half of the cells of the monolayer are preserved and frozen.

5. The method of claim 4, wherein the nucleic acid sample is purified from the unfrozen cells of the monolayer.

6. The method of claim 5, wherein microarray assays are used to generate the genetic data.

7. The method of claim 5, wherein antisense is produced for each nucleic acid associated with the specific gene-drug response.

8. The method of claim 7, wherein the antisense is used as a reagent on cells that are representative of the tissue sample of the proliferating cells.

9. The method of claim 8, wherein the antisense reagent is used in a micro version cell-based patient assay system.

10. The method of claim 8, wherein the representative cells are determined based on fluorescent staining for cell types and subtypes.

11. The method of claim 4, wherein the portion of the monolayer is preserved from a group of preservatives consisting of DMSO, glycerol and mannitol.

12. The method of claim 4, wherein the portion of the monolayer is frozen in liquid nitrogen.

13. The method of claim 4, wherein the nucleic acid sample is purified from the frozen cells of the monolayer.

14. The method of claim 13, wherein microarray assays are used to generate the genetic data.

15. The method of claim 13, wherein antisense is produced for each nucleic acid associated with the specific gene-drug response.

16. The method of claim 15, wherein the antisense is used as a reagent on cells that are representative of the tissue sample of the proliferating cells.

17. The method of claim 16, wherein the antisense reagent is used in a micro version cell-based patient assay system.

18. The method of claim 16, wherein the representative cells are determined based on fluorescent staining for cell types and subtypes.

19. The method of claim 18, wherein the gene relationship database structure, the micro version cell-based patient assay system, and the dynamic assays from the unfrozen cells of the monolayer are compared to the gene relationship database structure, the micro version cell-based patient assay system, and the dynamic assays from the frozen cells of the monolayer.

20. An assay method for characterizing proliferating cells, comprising the steps of:
a. culturing proliferating cells by:
i. collecting a tissue sample including the proliferating cells;
ii. mechanically dividing the sample into cohesive multicellular particulates; and
iii. growing a tissue culture monolayer from the multicellular particulates;
b. purifying nucleic acids from cells of the monolayer; and
c. analyzing the nucleic acid using a method for characterizing the nucleic acid of proliferating cells, thereby generating genetic data specific to the tissue sample.

21. The method of claim 20, in which the nucleic acid is DNA.

22. The method of claim 20, in which the nucleic acid is RNA.

23. The method of claim 20, in which the method for characterizing the nucleic acid includes the step of sequencing at least a portion of one of the nucleic acids.

24. The method of claim 20, in which the nucleic acid is analyzed for polymorphisms characteristic of a proliferative state.

25. The method of claim 20, in which the method for characterizing the proliferating cells includes the step of determining the relative amount of a specific RNA as compared to the amount of the same RNA in a non-proliferating cell.

26. The method of claim 20, comprising the additional step of phenotypically analyzing the cells.

27. The method of claim 20, further comprising the step of characterizing the proliferating cells by analyzing a set of genetic data in connection with a set of corresponding clinical data for statistically significant commonalities and/or trends to generate one or more profiles which links one or more proliferative cell disease states with phenotypic and/or genotypic characterizations, diagnoses and/or prognoses.

28. The method of claim 27, further comprising the step of encoding the one or more profiles in a computer storage medium.

29. The method of claim 28, further comprising the step of providing access to profiles over a computer network.

30. The method of claim 20, further comprising the step of encoding the genetic data in a computer storage medium in connection with corresponding clinical data.

31. The method of claim 30, further comprising the step of analyzing a set of the encoded genetic data in connection with a set of corresponding clinical data for statistically significant commonalities and/or trends to generate one or more profiles which links one or more proliferative cell disease states with phenotypic and/or genotypic characterizations, diagnoses and/or prognoses.

32. The method of claim 31, further comprising the step of encoding the one or more profiles in a computer storage medium.

33. The method of claim 32, further comprising the step of comparing genetic data derived from a tissue sample from a patient with the encoded profiles to provide a patient-specific diagnosis or prognosis.

34. The method of claim 32, further comprising the steps of adding to the encoded sets of genetic and corresponding clinical data an additional set of genetic and corresponding clinical data and incorporating the data into the profiles.

35. The method of claim 32, further comprising the step of providing access to the set of genetic data, the set of corresponding clinical data and/or profiles over a computer network.

36. The method of claim 31, further providing a means for adding data generated by the analyzing step to the set of encoded genetic data over a computer network.

37. The method of claim 30, in which the clinical data includes data indicating to what treatment(s) the patient from which the tissue sample was taken was subjected.

38. The method of claim 37, in which the clinical data includes data indicating the clinical result of the treatment(s).

39. The method of claim 30, further comprising the step of providing access to the set of genetic data and/or corresponding clinical data over a computer network.

40. The method of claim 29, in which the method for characterizing the proliferating cells is performed using a nucleic acid microarray.

41. The method of claim 20, in which the method for characterizing the nucleic acid of proliferating cells is performed using a molecular beacon allelic discrimination probe.

42. The method of claim 20, in which the analyzing step is at least partially automated.

43. A method for preparing a diagnostic database for proliferative diseases comprising the steps of:
a. culturing proliferating cells by the steps of:
i. collecting a tissue sample including the proliferating cells;
ii. mechanically dividing the sample into cohesive multicellular particulates; and
iii. growing a tissue culture monolayer from the multicellular particulates;
b. purifying a nucleic acid sample from cells of the monolayer;
c. analyzing the nucleic acid using a method for characterizing the nucleic acid of proliferating cells, thereby generating genetic data specific to the tissue sample;
d. encoding the genetic data generated by the analyzing step onto a computer storage medium so that the data is retrievable and searchable as part of a database;
e. encoding corresponding clinical data specific to the patient from whom the tissue sample was collected onto a computer storage medium so that the data is retrievable and searchable as part of a database, wherein the clinical data specific to each patient is linked to the data generated by the analyzing step for a tissue sample from the same patient; and
f. providing a database search engine or a data mining engine that allows searching, retrieval and/or analysis of either or both of the genetic data and the clinical data.

44. A computer device useful for detecting and analyzing proliferating cells, comprising:
a. a set of genetic data encoded in a computer storage medium so that the data is retrievable and searchable as part of a database, the genetic data being generated by the steps of:
i. culturing proliferating cells by:
1. collecting a tissue sample including the proliferating cells;
2. mechanically dividing the sample into cohesive multicellular particulates; and
3. growing a tissue culture monolayer from the multicellular particulates;
ii. purifying a nucleic acid sample from cells of the monolayer; and
iii. analyzing the nucleic acid using a method for characterizing the nucleic acid of the proliferating cells; and
b. a set of clinical data encoded in a computer storage medium so that the data is retrievable and searchable as part of a database, the clinical data corresponding to the genetic data, wherein the genetic data is limited to the corresponding clinical data.

45. A method for characterizing a proliferative disease in a patient, comprising the steps of:
a. culturing proliferating cells by:
i. collecting a tissue sample including the proliferating cells;
ii. mechanically dividing the sample into cohesive multicellular particulates; and
iii. growing a tissue culture monolayer from the multicellular particulates;
b. purifying a nucleic acid sample from cells of the monolayer;
c. analyzing the nucleic acid using a method for characterizing the nucleic acid of the proliferating cells, thereby generating genetic data specific to the tissue sample; and
d. comparing the genetic data and, optionally, corresponding clinical data to profiles that link genetic and corresponding clinical data to known proliferative disease states, thereby identifying a disease state having the best matching genetic and, optionally, clinical data.

46. The method of claim 45, in which the profiles are encoded in a computer storage medium so that the profiles are retrievable and searchable as part of a database, the comparing step being performed by entering clinical data for a tissue sample into software that compares the input data to the profiles encoded in the computer storage medium.

47. A method for distributing information characterizing a proliferative disease, comprising the step of providing access over a computer or computer network to data generated by a cell culture method comprising the steps of:
a. culturing proliferating cells by:
i. collecting a tissue sample including the proliferating cells;
ii. mechanically dividing the sample into cohesive multicellular particulates; and
iii. growing a tissue culture monolayer from the multicellular particulates;
b. purifying a nucleic acid sample from cells of the monolayer;
c. analyzing the nucleic acid using a method for characterizing the nucleic acid of the proliferating cells, thereby generating genetic data specific to the tissue sample; and
d. encoding the genetic data in a computer storage medium that is accessible from a computer or over a computer network so that the data are retrievable and searchable as part of a database.
